(19) United States
(12) Patent Application Publication
(10) Pub. No.: US 2005/0043333 A1
(43) Pub. Date: Feb. 24, 2005

(54) Quinazolinone derivative

(75) Inventors: Junya Ishida, Osaka (JP); Kouji Hattori, Osaka (JP); Yoshiyuki Kido, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Fujisawa Pharmaceutical Co., Ltd, Osaka (JP)

(21) Appl. No.: 10/499,348

(22) PCT Filed: Dec. 19, 2002
(86) PCT No.: PCT/JP02/13286

(30) Foreign Application Priority Data
Dec. 24, 2001 (AU) PR9756

(51) Int. Cl. A61K 31/517; C07D 43/02

(52) U.S. Cl. 514/266.2; 544/284

(57) ABSTRACT

A quinazolinone derivatives having poly (adenosine 5′-diphospho-ribose) polymerase (PARP) inhibitory activity represented by the formula (I): wherein R¹ is substituted cyclic amino groups or optionally substituted amino group, R² is substituent, n means an integer of 0 to 4, and L¹ is (1) cyclo (lower) alkylene, (2) cyclo (lower) alkenylene, (3) diradical of saturated- or unsaturated monocyclic group with one or more nitrogen atom(s), which is obtained after removal of one hydrogen atom from said monocyclic group, or (4) —N(R³)—L²— (wherein R³ is hydrogen or lower alkyl, and L² is lower alkylene or lower alkenylene), or its prodrug, or a salt thereof.

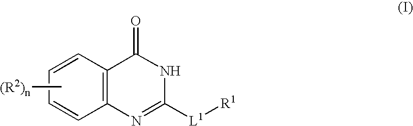

### TECHNICAL FIELD

This invention relates to a novel quinazolinone derivative having pharmacological activity, to a process for their production and to a pharmaceutical composition containing the same.

### BACKGROUND ART

Poly(adenosine 5′-diphospho-ribose)polymerase (“poly(ADP-ribose)polymerase” or “PARP”, which is also sometimes called “PARS” for “poly(ADP-ribose)synthetase”] is an enzyme located in the nuclei of cells of various organs, including muscle, heart and brain cells. PARP plays a physiological role in the repair of strand breaks in DNA. Once activated by damaged DNA fragments, PARP catalyzes the attachment of up to 100 ADP-ribose units to a variety of nuclear proteins, including histones and PARP itself.

Some quinazolinone derivatives having inhibitory activity of PARP have been known, for example, in WO95/24379, WO98/33802 and WO99/11624.

### DISCLOSURE OF THE INVENTION

This invention relates to a novel quinazolinone compound, which has pharmaceutical activity such as PARP inhibiting activity, to a process for their production, to a pharmaceutical composition containing the same and to a use thereof.

One object of this invention is to provide the novel quinazolinone compound, which has a PARP inhibiting activity.

Another object of this invention is to provide a process for production of the quinazolinone compound.

A further object of this invention is to provide a pharmaceutical composition containing the quinazolinone compound as an active ingredient.

Still further object of this invention is to provide a use of the quinazolinone compound for manufacturing a medicament for treating or preventing various diseases, or a method of treating or preventing various diseases by administering the quinazolinone compound in an effective amount to inhibit PARP activity.

The quinazolinone compound of this invention can be represented by the following formula (I): 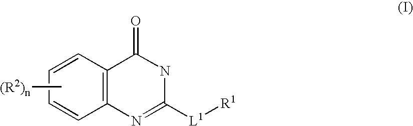
[wherein R¹ is substituted cyclic amino groups, optionally substituted carbocyclic group or optionally substituted amino group,
R² is substituent,
n means an integer of 0 to 4, and
L¹ is (1) cyclo(lower)alkylene, (2) cyclo(lower)alkenylene, (3) diradical of saturated- or unsaturated monocyclic group with one or more nitrogen atom(s), which is obtained after removal of one hydrogen atom from said monocyclic group, or (4) —N(R³)—L²— (wherein
R³ is hydrogen or lower alkyl, and L² is lower alkylene or lower alkenylene)], or its prodrug, or a salt thereof.

The compound (I) or its prodrug, or a salt thereof can be prepared by the following processes. In the following formulae, the compounds may be prodrugs or their salts.
Process 1 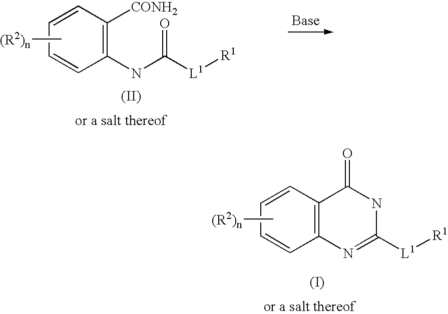
[wherein, R¹, R², n and L¹ are each as defined above.]

In this process, the compound (I) or a salt thereof can be produced by subjecting the compound (II) to cyclization reaction in the presence of base, such as inorganic bases, for example, an alkali metal [e.g., sodium or potassium], alkoxide, hydroxide, carbonate or bicarbonate thereof, or organic bases such as a trialkylamine [e.g., trimethylamine or triethylamine] or the like.

The reaction is usually carried out in a conventional solvent such as water, an alcohol (e.g., methanol, ethanol or isopropyl alcohol), ether, tetrahydrofuran, dioxane, diethylether, amide (e.g., N,N-dimethylformamide, N,N-dimethylacetamide), nitrile (e.g., acetonitrile), or any other organic solvent which does not adversely affect the reaction. There action may be usually carried out under cooling to heating since the reaction temperature is not critical.
Process 2 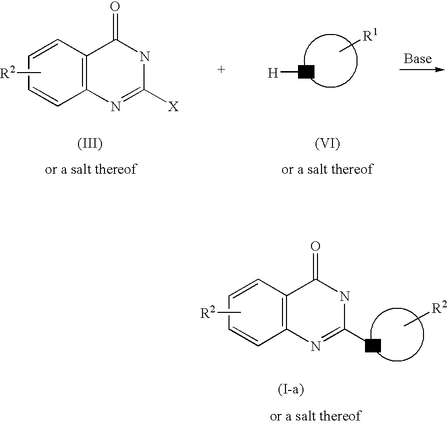
[wherein, X is leaving group, 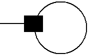
is saturated- or unsaturated monocyclic group with one or more nitrogen atom(s), and R¹ and n are each as defined above.]

In this process, the compound (I-a) or a salt thereof can be produced by reacting the compound (III) or a salt thereof and compound (VI) or a salt thereof in the presence of base, such as inorganic bases, for example, an alkali metal [e.g., sodium or potassium], alkoxide, hydroxide, carbonate or bicarbonate thereof, or organic bases such as a trialkylamine [e.g., trimethylamine or triethylamine] or the like.

The reaction is usually carried out in a conventional solvent such as an alcohol (e.g., methanol, ethanol or isopropyl alcohol), tetrahydrofuran, dioxane, diethylether, amide (e.g., N,N-dimethylformamide, N,N-dimethylacetamide), nitrile (e.g., acetonitrile), or any other organic solvent which does not adversely affect the reaction. The reaction may be usually carried out under cooling to heating since the reaction temperature is not critical.

The compounds of the present invention can be purified by any conventional purification methods employed for purifying organic compounds, such as recrystallization, column chromatography, thin-layer chromatography, high-performance liquid chromatography and the like. The compounds can be identified by conventional methods such as NMR spectrography, mass spectrography, IR spectrography, elemental analysis, and measurement of melting point.

Some of the starting compounds (II) or a salt thereof are novel and can be prepared by the well-known processes or its analogous processes, for example, the processes described in the J. Med. Chem. 1998, 41, 5247-5256 and J. Org. Chem., 21, 478-(1956). The following process is given as an example.
Reference Process 1 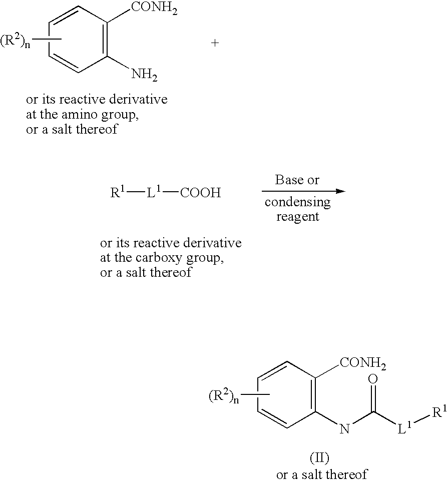
[wherein, R¹, R², n and L¹ are each as defined above.]

Suitable salt of the compound (I) of the present invention are pharmaceutically acceptable conventional non-toxic salts and can be an organic acid addition salt (e.g. formate, acetate, trifluoroacetate, maleate, tartrate, oxalate, methanesulfonate, benzenesulfonate, toluenesulfonate, etc.), an inorganic acid addition salt (e.g. hydrochloride, hydrobromide, sulfate, phosphate, etc.), a salt with an amino acid (e.g. aspartic acid salt, glutamic acid salt, etc.), or the like.

The “prodrug” means the derivative of compound of the present invention having a chemically or metabolically degradable group, which becomes pharmaceutically active after biotransformation.

The compound (I) of the present invention may contain one or more asymmetric centers and thus they can exist as enantiomers or diastereoisomers. Further more certain the compound (I) which contains alkenyl groups may exist as cis- or trans-isomers. In each instance, the invention includes both mixtures and separate individual isomers.

The compound (I) may also exist in tautomeric forms, and the invention includes both mixtures and separate individual tautomers.

The compound (I) or a salt thereof can be in a form of a solvate, which is included within the scope of the present invention. The solvate preferably includes a hydrate or an ethanolate.

The radiolabelled derivative of the compound (I), which is suitable for biological studies, may be included in the scope of invention.

In the above and subsequent description of the present specification, suitable examples and illustrations of the various definitions, which the present invention includes within the scope thereof, are explained in detail as follows.

The term “lower” means a group having 1 to 6 carbon atom(s), unless otherwise provided.

Suitable “lower alkyl” includes a straight or branched alkyl having 1 to 6 carbon atom(s), in particular 1 or 2 carbon atom(s).

Preferable examples which may be mentioned are methyl, ethyl, propyl, isopropyl, butyl, isobutyl, tert-butyl, pentyl and hexyl.

Suitable “lower alkoxy” includes straight or branched alkoxy having 1 to 6 carbon atom(s), in particular 1 or 2 carbon atom(s). Preferable examples which may be mentioned are methoxy, ethoxy, n-propoxy, isopropoxy, n-butoxy, iso-butoxy, sec-butoxy and tert-butoxy, preferably methoxy.

Suitable “lower alkylamino” include mono(lower)alkylamino and di(lower)alkylamino. Preferable examples which may be mentioned are methylamino, dimethylamino, ethylamino, dimethylamino, n-propylamino, isopropylamino, n-butylamino, iso-butylamino, sec-butylamino and tert-butylamino, preferably dimethylamino and diethylamino.

Suitable “aryl” may be intended to mean a mono-, di- or polynuclear aromatic radical having preferably 6 to 12 carbon atoms, such as phenyl, naphthyl, tetrahydronaphthyl, indenyl, indanyl (1,2-dihydroindenyl), fluorenyl and the like, preferably phenyl or naphthyl.

The term “halogen” means fluoro, chloro, bromo or iodo.

Suitable “halo(lower)alkyl” contains 1 to 4 carbon atom(s), in particular 1 or 2 carbon atom(s), and 1 to 9 halogen atom(s), in particular 1 to 5 identical or different halogen atom(s), preferably fluorine, chlorine and bromine, in particular fluorine and chlorine. Preferable examples which maybe mentioned are trifluoromethyl, trichloromethyl, chlorodifluoromethyl, dichlorofluoromethyl, chloromethyl, bromomethyl, 1-fluoroethyl, 2-fluoroethyl, 2,2-difluoroethyl, 2,2,2-trifluoroethyl, 2,2,2-trichloroethyl and pentafluoroethyl, preferably trifluoromethyl.

The term “carbocyclic group” is intended to mean cyclo(lower)alkyl or cyclo(lower)alkenyl.

Suitable “cyclo(lower)alkyl” and “cyclo(lower)alkyl moiety” in the term “cyclo(lower)alkylene” includes a saturated carbocycle having 3 to 7 carbon atoms, in particular 5 to 6 carbon atoms. Preferable examples which may be mentioned are cyclopropyl, cyclobutyl, cyclopentyl, cyclohexyl and cycloheptyl, preferably cyclopropyl and cyclohexyl (e.g., 1,3-cyclohexylene, 1,4-cyclohexylene, etc.).

Suitable “cyclo(lower)alkenyl” and “cyclo(lower)alkenyl moiety” in the term “cyclo (lower) alkenylene” includes a partially saturated carbocycle having 3 to 7 carbon atoms, in particular 5 to 6 carbon atoms. Preferable examples which may be mentioned are cyclopropenyl, cyclobutenyl, cyclopentenyl, cyclohexenyl and cycloheptenyl, preferably cyclopentenyl and cyclohexenyl.

Preferable example which may be mentioned as “cyclo(lower)alkylene” are cyclopentenylene (e.g., 1,3-cyclocyclopent-1-enylene, etc.), cyclohexenylene (e.g., 1,3-cyclohex-1-enylene, etc.).

Suitable “heteroaryl” and “heteroaryl” moiety in the terms “heteroaryl(lower)alkyl” and “heteroaromatic acyl” is intended to mean 5- to 7-membered rings having preferably 1 to 3 heteroatom(s), in particular 1 or 2 identical or different heteroatom(s). Heteroatoms in the heteroaryl are oxygen, sulfur or nitrogen. Examples which may be mentioned are furyl, thienyl, pyrazolyl, imidazolyl, triazolyl (e.g., 1,2,3- and 1,2,4-triazolyl, etc.), isoxazolyl, thiazolyl, isothiazolyl, oxadiazolyl (e.g., 1,3,4-, and 1,2,5-oxadiazolyl, etc.), azepinyl, pyrrolyl, pyridinyl, piperazinyl, pyridazinyl, pyrimidinyl, pyrazinyl, triazinyl (e.g., 1,3,5-, 1,2,4- and 1,2,3-triazinyl, etc.), oxazinyl (e.g., 1,2,4- and 1,2,6-oxazinyl, etc.), oxepinyl, thiepinyl and diazepinyl (e.g., 1,2,4-diazepinyl, etc.), preferably thienyl, pyrazolyl, imidazolyl, thiazolyl, pyridinyl and pyrazinyl.

Suitable “cyclic amino group” are heteroaromatic or aliphatic ring systems having one or more nitrogen atoms as the heteroatom, in which the heterocyclic rings can be saturated or unsaturated, can be one ring system or several fused ring systems, and optionally contain further heteroatoms, such as nitrogen, oxygen and sulfur and the like. Cyclic amino groups can furthermore also denote a spiro ring or a bridged ring system. The number of atoms which form cyclic amino groups is not limited, for example in the case of a single-ring system, they comprise 3 to 8 atoms, and in the case of a three-ring system, they comprise 7 to 11 atoms.

Preferable examples of “cyclic amino group” are described as follows:
(1) examples which may be mentioned of cyclic amino group with saturated monocyclic groups with one or more nitrogen atom(s) as the heteroatom are azetidinyl (3-azetidinyl), pyrrolidinyl (e.g., 1- and 3-pyrrolidinyl, etc.), piperidyl (e.g., 1- and 4-piperidyl, etc.), homopiperidino (e.g., hexahydro-1H-azepin-1-yl, etc.), homopiperazinyl (e.g., hexahydro-1H-1,4-diazepin-1-yl, etc.), imidazolidinyl (e.g., 1-imidazolidinyl, etc.), piperazinyl (e.g., 1-piperazinyl, etc.), perhydropyrimidinyl (e.g., perhydropyrimidin-1-yl, etc.) and diazacycloheptanyl (e.g., 1,4-diazacycloheptan-1-yl, etc.);
(2) examples which may be mentioned of cyclic amino group with unsaturated monocyclic groups with one or more nitrogen atom(s) as the heteroatom are pyrrolinyl (e.g., 2-pyrrolin-1-yl, etc.), pyrrolyl (e.g., 1-pyrrolyl, etc), tetrahydropridinyl (e.g., 3,6-dihydro-1 (2H)-pyridinyl, etc.), pyridinyl (e.g., 2-pyridinyl, etc.), tetrahydroazepinyl (e.g., 2,3,6,7-tetrahydro-1H-azepin-1-yl, 2,3,4,7-tetrahydro-1H-azepin-1-yl, etc.), imidazolyl (1-imidazolyl), pyrazolyl, triazolyl, tetrazolyl, tetrazolyl, pyrimidinyl, pyrazinyl, pyridazinyl, dihydro-pyridazinyl (e.g., 1,2-dihydro-pyridazin-1-yl, etc.) and dihydro-pyrimidinyl (e.g., 1,2-dihydro-pyrimidin-1-yl, etc.);
(3) examples which may be mentioned of cyclic amino groups with saturated and unsaturated monocyclic groups with 1 to 3 nitrogen atom(s) and 1 or 2 sulfur atom(s) as heteroatoms are thiazolidinyl (e.g., 3-thiazolidinyl, etc.), isothiazolinyl (e.g., 2-isothiazolinyl, etc.) and thiomorpholino;
(4) examples which may be mentioned of cyclic amino groups with saturated and unsaturated monocyclic groups with 1 to 3 nitrogen atom(s) and 1 or 2 oxygen atom(s) as heteroatoms are oxazolyl, isoxazolyl, oxadiazolyl (e.g., 1,2,4-oxadiazolyl, and 1,3,4-oxadiazolyl) or morpholinyl;
(5) examples which may be mentioned of cyclic amino groups with saturated and unsaturated fused cyclic groups are indolyl (e.g., 1-indolyl, etc.)., dihydrobenzimidazolyl (e.g., 1,2-dihydrobenzimidazol-1-yl, etc.), perhydropyrrolo[1,2-a]pyrazinyl (e.g., perhydropyrrolo[1,2-a]pyrazin-2-yl, etc.), tetrahydrobenzo[f]isoquinolinyl (e.g., 1,4,5,6-tetrahydrobenzo[f]isoquinolin-3(2H)-yl, etc.), hexahydrobenz[f]isoquinolinyl (e.g., cis- and trans-1,4,4a,5,6,10b-hexahydrobenz[f]isoquinolin-3(2H)-yl, etc.), tetrahydropyrido[3,4-b]indolyl (e.g., 1,3,4,9-tetrahydro-2H-pyrido[3,4-b]indol-2-yl, etc.) tetrahydrobenzazepinyl (e.g., 1,2,4,5-tetrahydro-3H-3-benzazepin-3-yl, etc.) dihydroisoquinolinyl (e.g., 3,4-dihydro-2(1H)-isoquinolinyl, etc.);
(6) examples which may be mentioned of cyclic amino groups with spirocyclic groups are azaspiro[4,5]decanyl (e.g., 2-azaspiro[4,5]decan-2-yl, etc.), spiro[1H-indene-1,4′-piperidinyl] (e.g., spiro[1H-indene-1,4′-piperidin-1′-yl], etc.), and dihydrospiro[1H-indene-1,4′-piperidinyl] (e.g., 2,3-dihydrospiro[1H-indene-1,4′-piperidin-1′-yl], etc.);
   (7) examples which may be mentioned of cyclic amino groups bridged heterocyclic groups are azabicyclo[2,2,1]heptanyl (e.g., 2-azabicyclo[2,2,1]heptan-7-yl, etc.) and diazabicyclo[2.2.1]heptyl (e.g., 2,5-diazabicyclo[2.2.1]hept-2-yl, etc.).

Among the above, preferable “cyclic amino group” included in R¹ is above-mentioned (1) or (2), in which the most preferable one may be piperidinyl, tetrahydropyridinyl and piperazinyl.

Preferable examples which may be mentioned of “diradical of saturated or unsaturated monocyclic group with one or more nitrogen atom(s), which is obtained after removal of one hydrogen atom from said monocyclic group” are azetidinylene (e.g., 1,2- or 1,3-azetidinylene), pyrrolidinylene (e.g., 1,2- or 1,3-pyrrolidinylene), piperidinylene (e.g., 1,3- or 1,4-piperidinylene).

It has been known that, during major cellular stresses, the activation of PARP can rapidly lead to cell damage or death through depletion of energy stores and PARP activation play a key role in both NMDA- and NO-induced neurotoxicity (Zhang et. al., Science, 263: 687-89 (1994)). Therefore, the compound possessing PARP inhibiting activity, such as the compound (I) or a pharmaceutically acceptable salt thereof of this invention is useful in treating and preventing various diseases ascribed by NMDA- and NO-induced toxicity. Such diseases include, for example, tissue damage resulting from cell damage or death due to necrosis or apoptosis; neural tissue damage resulting from ischemia and reperfusion injury, neurological disorders and neurodegenerative diseases; neurodegenerative diseases; head trauma; stroke; Alzheimer's disease; Parkinson's disease; epilepsy; Amyotrophic lateral Scleosis (ALS); Huntington's disease; schizophrenia; chronicpain; ischemia and neuronal loss following hypoxia; hypoglycemia; ischemia; trauma; and nervous insult.

It has been demonstrated that PARP inhibitor are useful in deducing infarct size (Thiemermann et al, Proc. Natl. Acad. Sci. USA, 94: 679-83 (1997)). Therefore, the compound possessing PARP inhibiting activity, such as the compound (I) orapharmaceutically acceptable salt thereof of this invention is useful in treatment and prevention of previously ischemic heart or skeleton muscle tissue.

It is also known that PARP is thought to play a role in enhancing DNA repair. So, the compound possessing PARP inhibiting activity, such as the compound (I) or a pharmaceutically acceptable salt thereof of this invention is effective in treating and preventing radiosensitizing hypoxic tumor cells; tumor cells from recovering from potentially lethal damage of DNA after radiation therapy.

Further, the compound possessing PARP inhibiting activity, such as the compound (I) or a pharmaceutically acceptable salt thereof of this invention is useful in extending the life-span and proliferative capacity of cells and altering gene expression of senescent cells. It is useful for treating and preventing skin aging; Alzheimer's diseases; arteriosclerosis; osteoarthritis; osteoporosis; muscular dystrophy; degenerative diseases of skeletal muscle involving replicative senescence; age-related macular degeneration; immune senescence; AIDS; and other immune senescence diseases.

Still further, the compound possessing PARP inhibiting activity, such as the compound (I) or a pharmaceutically acceptable salt thereof of this invention is effective in treating and preventing inflammatory bowel disorders (e.g., colitis); arthritis; diabetes; endotoxic shock; septic shock; and tumor. Also, it is useful in reducing proliferation of tumor cells and making synergistic effect when tumor cells are co-treated with an alkylamine drug.

The compound possessing PARP inhibiting activity, such as the compound (I) of this invention or a pharmaceutically acceptable salt thereof of this invention is effective in treating and preventing pituitary apoplexy; conjunctivitis; retinoblastoma; retinopathy; acute retinal necrosis syndrome; Sjogren's syndrome.

The compound (I), its prodrug, or a salt thereof can be administered alone or in the form of a mixture, preferably, with a pharmaceutical vehicle or carrier.

The active ingredient of this invention can be used in the form of a pharmaceutical preparation, for example, in solid, semisolid or liquid form, which contains a compound (I), as an active ingredient, in admixture with an organic or inorganic carrier or excipient suitable for external (topical), enteral, intravenous, intramuscular, parenteral or intramucous applications. The active ingredient can be formulated, for example, with the conventional non-toxic, pharmaceutically acceptable carriers for ointment, cream, plaster, tablets, pellets, capsules, suppositories, solution (saline, for example), emulsion, suspension (olive oil, for example), aerosols, pills, powders, syrups, injections, troches, cataplasms, aromatic waters, lotions, buccal tablets, sublingual tablets, nasal drops and any other form suitable for use. The carriers which can be used are water, wax, glucose, lactose, gum acacia, gelatin, mannitol, starch paster, magnesium trisilicate, talc, corn starch, keratin, paraffin, colloidal silica, potato starch, urea and other carriers suitable for use in manufacturing preparations, in solid, semisolid, or liquid form, and in addition auxiliary, stabilizing, thickening and coloring agents and perfumes may be used. The active compound is included in a pharmaceutical composition in an effective amount sufficient to produce the desired effect upon the process or condition of the diseases.

The active ingredient can be formulated into, for example, preparations for oral application, preparations for injection, preparations for external application, preparations for inhalation, preparations for application to mucous membranes.

Mammals which may be treated by the present invention include livestock mammals such as cows, horses, etc., domestic animals such as dogs, cats, rats, etc. and humans, preferably humans.

While the dosage of therapeutically effective amount of the compound (I) will vary depending upon the age and condition of each individual patient, an average single dose to a human patient of about 0.01 mg, 0.1 mg, 1 mg, 10 mg, 50 mg, 100 mg, 250 mg, 500 mg, and 1000 mg of the compound (I) may be effective for treating the above-mentioned diseases. In general, amounts between 0.01 mg/body and about 1,000 mg/body may be administered per day.

In order to illustrate the usefulness of the object compound (I), the pharmacological test data of the compound (I) are shown in the following.

(1) Test Compound:

Compound A:

2-[3-(4-phenyl-3,6-dihydro-1(2H)-pyridinyl)-1-cyclopenten-1-y 1]-4(3H)-quinazolinone (The compound of Example 2-4)

Compound B:

2-[4-(4-Phenyl-3,6-dihydro-1(2H)-pyridinyl)-1-piperidinyl]-4(3H)-quinazolinone (The compound of Example 6-3)

PARP Inhibitory Activity (In Vitro Assay)

(2) Assay Conditions:

The recombinant human PARP (5.3 mg protein/ml) were incubated with a test compound in a 100 μl reaction buffer containing the indicated concentration of 1 mCi/ml ³²P-NAD, 50 mM Tris-HCl, 25 mM MgCl₂, 1 mM DTT (dithiothreitol), 0.05 mM NAD (nicotinamido adenine dinucleotide), 1 mg/ml activated DNA, pH8.0. Incubation was for 15 minutes at a room temperature and the reaction was stopped by the addition of 200 μl of ice-cold 20% trichloroacetic acid followed by rapid filtration through GF/B filters. The filters were treated with scintillation fluid and acid-insoluble counts were measured for quantification of unit activity.
PARP inhibitory activity (%)=[1−(enzyme activity with test compound)/(enzyme activity with vehicle)]×100
(3) Result

PARP inhibitory activity (IC₅₀) in test compound.

| Test Compound | IC50 (μM) |
|---|---|
| Compound A | <0.5 |
| Compound B | <0.5 |

This invention relates to novel Quinazolinone compounds had a potent PARP inhibitory activity. PARP inhibitors including this invention relates to novel quinazolinone compounds were effective in preventing reduction of striate DA and its metabolite induced by MPTP treatment in mice. Therefore, it suggests that these compounds may have protective benefit in the treatment of neurodegenerative disease such as Parkinson's disease.

Abbreviations used herein have the following meanings:

| ABBREVIATION | DEFINITION |
|---|---|
| Me | methyl |
| Et | ethyl |
| TBu | tert-buthyl |
| Bzl | benzyl |
| Ph | phenyl |
| Ac | acetyl |
| Bz | benzoyl |

Any patents, patent applications, and publications cited herein are incorporated by reference.

### BEST MODE FOR CARRYING OUT THE INVENTION

The following Preparation and Examples are given for the purpose of illustrating the present invention in detail, but are not to be construed to limit the scope of the present invention.

Preparation 1

N-ethyl-N,N-diisopropylamine (0.174 mL, 1.00 mmol) and O-(7-azabenzotriazol-1-yl)-N,N,N′,N′-tetramethyluronium hexafluorophosphate (380 mg, 1.00 mmol) were added to a solution of 2-aminobenzamide (136 mg, 1.00 mmol) and 1-(4-phenylcyclohexyl)-3-piperidinecarboxylic acid (287 mg, 1.00 mmol) in N,N-dimethylformamide (3 mL) at room temperature. The mixture was stirred at room temperature for 6 hours. Quenched with water, and the organic materials were extracted with chloroform. The crude product was washed with methanol and chloroform to give N-[2-(aminocarbonyl) phenyl]-1-(4-phenylcyclohexyl)-3-piperidinecarboxamide (188 mg, 46.4%) as product.

Mass (APCI): 405.93 (M⁺+H)

Preparation 2

The following compounds were prepared in a similar manner to that of Preparation 1.

(1) 2-({[3-(4-phenyl-3,6-dihydro-1(2H)-pyridinyl)-1-cyclohexen-1-yl]-carbonyl}amino) benzamide

Mass (API-ES): 402.3 (M⁺+H)

(2) 2-({4-[4-(3-methoxyphenyl)-3,6-dihydro-1(2H)-pyridinyl]butanoyl}-amino)benzamide

Mass (API-ES): 283.3 (M⁺+H)

(3) 2-({[3-(4-phenyl-1-piperidinyl)cyclohexyl]-carbonyl}amino)benzamide

Mass (APCI): 405.80 (M⁺+H)

Preparation 3

To a solution of 2-{[(4-oxocyclohexyl)carbonyl]amino}-benzamide (260 mg, 1.00 mmol) and 4-phenyl-1,2,3,6-tetrahydropyridine hydrochloride (293 mg, 1.50 mmol) in tetrahydrofuran (5 mL), sodium triacetoxyborohydride (318 mg, 1.50 mmol) and acetic acid (0.086 mL, 1.50 mmol) were added at room temperature. The mixture was stirred for 15 hours, and the reaction was quenched with water. The organic materials were extracted with chloroform and dried over sodium sulfate. Purification over silica gel chromatography gave 2-({[4-(4-phenyl-3,6-dihydro-1(2H)-pyridinyl)cyclohexyl]carbonyl}amino)benzamide (266 mg, 66.0%) as product.

Mass (API-ES): 404.4 (M⁺+H)

### EXAMPLE 1

2-{[4-(4-Phenyl-3,6-dihydro-1(2H)-pyridinyl)butanoyl]amino}benzamide (475 mg, 1.31 mmol) was dissolved in dioxane (5 mL). An aqueous solution of sodium hydroxide (1M, 3.92 mL) was added to the solution at room temperature, and the mixture was stirred at that temperature for 15 hours. The organic materials were extracted with chloroform, and the organic layer was washed with water and dried over sodium sulfate. Purification over silica gel chromatography gave cis- or trans-2-{3-[4-phenyl-3,6-dihydro-1(2H)-pyridinyl]propyl}-4(3H)-quinazolinone.

Less polar product (37 mg, 38.7%)

¹H NMR (200 MHz, DMSO-d₆, δ): 1.4-1.8 (4H, m), 1.9-2.2 (4H, m), 2.3-2.4 (1H, m), 2.6-2.8 (4H, m), 3.0-3.2 (3H, m), 6.19 (1H, br s), 7.2-7.5 (6H, m), 7.62 (1H, d, J=7.4 Hz), 7.75 (1H, t, J=8.3 Hz), 8.07 (1H, d, J=6.6 Hz), 12.08 (1H, br s).

Polar one product (30 mg, 31.4%)

¹H NMR (200 MHz, DMSO-d₆, δ): 1.2-1.8 (4H, m), 1.8-2.2 (4H, m), 2.4-2.6 (1H, m), 2.75 (2H, t, J=5.4 Hz), 3.0-3.3 (3H, m), 6.17 (1H, br s), 7.1-7.5 (6H, m), 7.59 (1H, d, J=7.8 Hz), 7.77 (1H, t, J=7.6 Hz), 8.08 (1H, d, J=7.9 Hz), 12.12 (1H, br s)

### EXAMPLE 2

The following compounds were prepared in a similar manner to that of Example 1.

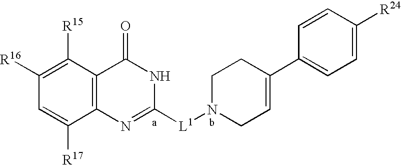

| | R¹⁵ | R¹⁶ | R¹⁸ | R²⁴ | (a)-L¹-(b) | |
|---|---|---|---|---|---|---|
| (1) | H | H | Me | F | 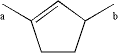 | ¹H NMR (200 MHz, CDCl₃; δ): 2.0-2.3 (2H, m), 2.62 (3H, s), 2.5-2.7 (2H, m), 2.8-3.0 (4H, m), 3.3-3.5 (2H, m), 4.18 (1H, m), 6.06 (1H, m), 6.9-7.7 (8H, m), 8.11 (1H, d, J=7.0 Hz) Mass: 402 (M⁺ + 1) |
| (2) | H | H | Cl | F | 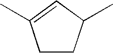 | ¹H NMR (200 MHz, CDCl₃: δ) 2.0-2.3 (2H, m), 2.5-2.7 (2H, m), 2.8-3.1 (4H, m), 3.3-3.5 (2H, m), 2.8-3.1 (4H, m), 3.3-3.5 (2H, m), 4.18 (1H, m), 6.06 (1H, m), 6.9-7.6 (6H, m), 7.81 (1H, dd, J=7.0 Hz), 8.12 (1H, d, J=7.0 Hz) Mass: 422 (M⁺ + 1) |
| (3) | H | H | Cl | H | 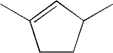 | ¹H NMR (200 MHz, CDCl₃: δ) 2.0-2.3 (2H, m), 2.5-2.7 (2H, m), 2.8-3.1 (4H, m), 3.3-3.5 (2H, m), 4.18 (1H, m), 6.11 (1H, m), 7.0-7.16 (7H, m), 7.82 (1H, dd, J=7.0 Hz), 8.12 (1H, d, J=7.0 Hz) Mass: 404 (M⁺ + 1) |
| (4) | H | H | H | H | 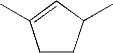 | ¹H NMR (200 MHz, CDCl₃: δ) 2.0-2.3 (2H, m), 2.5-2.7 (2H, m), 2.8-3.1 (4H, m), 3.3-3.5 (2H, m), 4.18 (1H, m), 6.17 (1H, m), 7.0-7.8 (9H, m), 8.27 (1H, d, J=7.0 Hz) Mass: 370 (M⁺ + 1) |
| (5) | H | H | H | F | 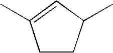 | ¹H NMR (200 MHz, CDCl₃: δ) 2.0-2.3 (2H, m), 2.5-2.7 (2H, m), 2.8-3.1 (4H, m), 3.3-3.5 (2H, m), 4.18 (1H, m), 6.05 (1H, m), 6.9-7.5 (6H, m), 7.75 (2H, m), 8.28 (1H, d, J=7.0 Hz) Mass: 388 (M⁺ + 1) |
| (6) | H | Me | H | F | 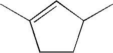 | ¹H NMR (200 MHz, CDCl₃: δ) 2.0-2.3 (2H, m), 2.41 (3H, s), 2.5-2.7 (2H, m), 2.8-3.1 (4H, m), 3.3-3.5 (2H, m), 4.20 (1H, m), 6.05 (1H, m), 6.9-7.7 (7H, m), 8.06 (1H, s) Mass: 402 (M⁺ + 1) |
| (7) | H | Cl | H | H | 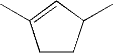 | ¹H NMR (200 MHz, CDCl₃: δ) 2.0-2.3 (2H, m), 2.5-2.7 (2H, m), 2.8-3.1 (4H, m), 3.3-3.5 (2H, m), 4.20 (1H, m), 6.07 (1H, m), 6.9-7.5 (5H, m), 7.69 (2H, s), 8.22 (1H, s) Mass: 422 (M⁺ + 1) |
| (8) | H | Cl | Cl | F | 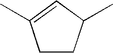 | ¹H NMR (200 MHz, CDCl₃: δ) 2.0-2.3 (2H, m), 2.5-2.7 (2H, m), 2.8-3.1 (4H, m), 3.3-3.5 (2H, m), 4.20 (1H, m), 6.05 (1H, m), 6.9-7.5 (5H, m), 7.89 (1H, s), 8.14 (1H, s) Mass: 457 (M⁺ + 1) |
| (9) | Cl | H | Cl | H | 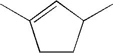 | ¹H NMR (200 MHz, CDCl₃: δ) 2.0-2.3 (2H, m), 2.5-2.7 (2H, m), 2.8-3.1 (4H, m), 3.3-3.5 (2H, m), 4.20 (1H, m), 6.02 (1H, m), 7.0-7.5 (6H, m), 7.71 (1H, d, J=8.4 Hz) Mass: 457 (M⁺ + 1) |
| (10) | H | H | H | H | 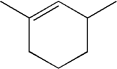 | ¹H NMR (400 MHz, DMSO-d₆: δ) 1.5-1.7 (2H, m), 1.84 (1H, br s), 1.96 (1H, br s), 2.35 (1H, br d, J=16.7 Hz), 2.69 (1H, d, J=17.3 Hz), 2.7-2.9 (2H, m), 3.2-3.4 (3H, m), 3.51 (1H, br s), 6.19 (1H, s), 6.98 (1H, s), 7.24 (1H, t, J=7.0 Hz), 7.34 (2H, t, J=7.6 Hz), 7.43 (2H, d, J=7.6 Hz), 7.48 (1H, t, J=7.5 Hz), 7.64 |
| # (1H, d, J=8.1 Hz), 7.79 (1H, t, J=7.7 Hz), 8.10 (1H, d, J=7.6 Hz), 12.15 (1H, br s). | | | | | | |

### EXAMPLE 3

The following compounds were prepared in a similar manner to that of Example 1.

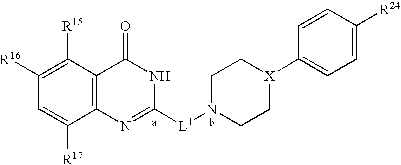

| | R¹⁵ | R¹⁶ | R¹⁸ | R²⁴ | (a)-L¹-(b) | X | |
|---|---|---|---|---|---|---|---|
| (1) | Cl | H | Cl | H | 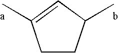 | CH | ¹H NMR (200 MHz, CDCl₃: δ) 1.4-2.5 (10H, m), 2.8-3.1 (3H, m), 4.11 (1H, m), 7.0-7.4 (9H, m), 7.71 (1H, d, J=8.4 Hz) Mass: 441 (M⁺ + 1) |
| (2) | H | Cl | Cl | H | 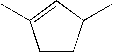 | CH | ¹H NMR (200 MHz, CDCl₃: δ) 1.4-2.5 (10H, m), 2.8-3.1 (3H, m), 4.09 (1H, m), 7.0-7.4 (8H, m), 7.81 (1H, m), 8.15 (1H, m) Mass: 441 (M⁺ + 1) |
| (3) | H | H | Cl | F | 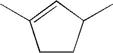 | CH | ¹H NMR (200 MHz, CDCl₃: δ) 1.8-2.1 (5H, m), 2.1-2.6 (4H, m), 2.8-3.3 (4H, m), 4.05 (1H, m), 6.9-7.4 (7H, m), 7.85 (1H, d, J=8 Hz), 8.19 (1H, d, J=8 Hz) Mass: 424 (M⁺ + 1) |
| (4) | H | H | Cl | Cl | 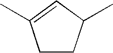 | CH | ¹H NMR (200 MHz, CDCl₃: δ) 1.8-2.1 (5H, m), 2.1-2.6 (4H, m), 2.8-3.3 (4H, m), 4.05 (1H, m), 6.9-7.4 (7H, m), 7.82 (1H, d, J=8 Hz), 8.19 (1H, d, J=8 Hz) Mass: 441 (M⁺ + 1) |
| (5) | H | H | Cl | H | 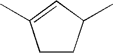 | CH | ¹H NMR (200 MHz, CDCl₃: δ) 1.8-2.1 (5H, m), 2.1-2.6 (4H, m), 2.8-3.3 (4H, m), 4.05 (1H, m), 7.0-7.4 (7H, m), 7.82 (1H, d, J=8 Hz), 8.20 (1H, d, J=8 Hz) Mass: 406 (M⁺ + 1) |
| (6) | H | H | Me | F | 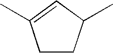 | CH | ¹H NMR (200 MHz, CDCl₃: δ) 1.8-2.1 (5H, m), 2.1-2.6 (4H, m), 2.8-3.3 (4H, m), 4.05 (1H, m), 6.8-7.4 (6H, m), 7.60 (1H, d, J=8 Hz), 8.14 (1H, d, J=8 Hz) Mass: 404 (M⁺ + 1) |
| (7) | H | H | Me | Cl | 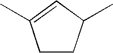 | CH | ¹H NMR (200 MHz, CDCl₃: δ) 1.8-2.1 (5H, m), 2.1-2.6 (4H, m), 2.8-3.3 (4H, m), 4.05 (1H, m), 6.8-7.4 (6H, m), 7.60 (1H, d, J=8 Hz), 8.14 (1H, d, J=8 Hz) Mass: 420 (M⁺ + 1) |
| (8) | H | Me | H | F | 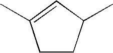 | CH | ¹H NMR (200 MHz, CDCl₃: δ) 1.8-2.1 (5H, m), 2.1-2.6 (4H, m), 2.44 (3H, s), 2.8-3.3 (4H, m), 4.05 (1H, m), 7.0-7.4 (6H, m), 7.5-7.7 (2H, m), 8.08 (1H, s) Mass: 386 (M⁺ + 1) |
| (9) | H | Cl | H | H | 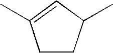 | CH | ¹H NMR (200 MHz, CDCl₃: δ) 1.8-2.1 (5H, m), 2.1-2.6 (4H, m), 2.44 (3H, s), 2.8-3.3 (4H, m), 4.05 (1H, m), 7.0-7.4 (6H, m), 7.6-7.7 (2H, m), 8.24 (1H, s) Mass: 406 (M⁺ + 1) |
| (10) | H | Cl | H | Cl | 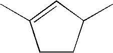 | CH | ¹H NMR (200 MHz, CDCl₃: δ) 1.8-2.1 (5H, m), 2.1-2.6 (4H, m), 2.44 (3H, s), 2.8-3.3 (4H, m), 4.05 (1H, m), 7.0-7.4 (5H, m), 7.6-7.8 (2H, m), 8.24 (1H, s) Mass: 441 (M⁺ + 1) |
| (11) | H | H | Me | Cl | 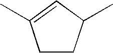 | N | ¹H NMR (200 MHz, CDCl₃: δ) 1.8-2.2 (2H, m), 2.62 (3H, m), 2.7-3.3 (12H, m), 4.08 (1H, m), 6.7-6.9 (3H, m), 7.1-7.3 (3H, m), 7.60 (1H, d, J=8 Hz), 8.13 (1H, d, J=8 Hz) Mass: 421 (M⁺ + 1) |
| (12) | H | H | Me | F | 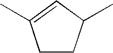 | N | ¹H NMR (200 MHz, CDCl₃: δ) 1.8-2.2 (2H, m), 2.62 (3H, m), 2.7-3.3 (12H, m), 4.06 (1H, m), 6.8-7.3 (6H, m), 7.59 (1H, d, J=8 Hz), 8.14 (1H, d, J=8 Hz) Mass: 405 (M⁺ + 1) |
| (13) | H | H | Cl | F | 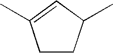 | N | ¹H NMR (200 MHz, CDCl₃: δ) 1.8-2.2 (2H, m), 2.7-3.3 (12H, m), 4.06 (1H, m), 6.8-7.3 (6H, m), 7.81 (1H, d, J=8 Hz), 8.21 (1H, d, J=8 Hz) Mass: 425 (M⁺ + 1) |
| (14) | H | H | H | Cl | 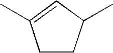 | N | ¹H NMR (200 MHz, CDCl₃: δ) 1.8-2.2 (2H, m), 2.62 (3H, m), 2.7-3.3 (12H, m), 4.08 (1H, m), 6.8-7.0 (2H, d, J=8 Hz), 7.09 (1H, m), 7.21 (2H, d, J=8 Hz), 7.45 (1H, t, J=8 Hz), 7.61 (1H, d, J=8 Hz), 7.75 (1H, t, J=8 Hz), 8.09 (1H, d, J=8 Hz) Mass: 407 (M⁺ + 1) |
| (15) | H | H | H | H | 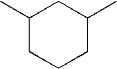 | CH | ¹H NMR (200 MHz, DMSO-d₆: δ) 1.4-2.3 (14H, m), 3.0-3.5 (5H, m), 7.1-7.4 (5H, m), 7.46 (1H, 5, J=7.6 Hz), 7.62 (1H, d, J=7.4 Hz), 7.78 (1H, t, J=7.6 Hz), 8.08 (1H, d, J=6.6 Hz), 12.12 (1H, br s) Mass (APCI) 387.73 (M⁺ + H) |

### EXAMPLE 4

The following compound was prepared in a similar manner to that of Example 1.

(1) 2-[1-(4-Phenylcyclohexyl)-3-piperidinyl]-4(3H)-quinazolinone

¹H NMR (200 MHz, CDCl₃: δ): 1.6-2.3 (13H, m), 2.4-2.6 (2H, m), 2.84 (1H, sept., J=3.8 Hz), 3.09 (1H, br s), 3.18 (1H, br d, J=10.7 Hz), 3.32 (1H, br d, J=11.9 Hz), 7.1-7.5 (6H, 10 m), 7.62 (1H, d, J=7.0 Hz), 7.71 (1H, t, J=6.8 Hz), 8.29 (1H, d, J=8.0 Hz), 12.87 (1H, br s) Mass (APCI): 388.20 (M⁺+H).

### EXAMPLE 5

Triethylamine (1.54 mL, 11.1 mmol) was added to a suspension of 2-chloro-4(3H)-quinazolinone (100 mg, 0.554 mmol) and 2-(4-phenyl-3,6-dihydro-1(2H)-pyridinyl)ethanamine dihydrochloride (229 mg, 0.831 mmol) in N,N-dimethylformamide (3 mL), and the mixture was heated at 100° C. for 3 hours. Cooled to room temperature, and the reaction were quenched with water, and the product was extracted with ethyl acetate. The organic layer was washed with water and dried over sodium sulfate. Purification over silica gel chromatography gave 2-{[2-(4-phenyl-3,6-dihydro-1(2H)-pyridinyl)ethyl]amino}-4(3H)-quinazolinone (76 mg, 39.6%) as product.

¹H NMR (400 MHz, DMSO-d₆: δ): 2.51 (2H, br s), 2.64 (2H, t, J=6.0 Hz), 2.71 (2H, t, J=5.6 Hz), 3.17 (2H, d, J=3.1 Hz), 3.51 (2H, q, J=5.5 Hz), 6.18 (1H, t, J=3.5 Hz), 6.36 (1H, br s), 7.10 (1H, t, J=7.5 Hz), 7.2-7.3 (2H, m), 7.34 (2H, t, J=6.5 Hz), 7.44 (2H, d, J=7.2 Hz), 7.56 (1H, t, J=7.7 Hz), 7.87 (1H, dd, J=7.9, 1.4 Hz), 11.05 (1H, br s)

### EXAMPLE 6

The following compounds were prepared in a similar manner to that of Example 5.

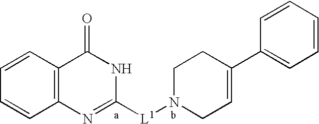

| | (a)-L¹-(b) | |
|---|---|---|
| (1) | 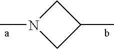 | ¹H NMR (400 MHz, DMSO-d₆: δ): 2.60 (2H, t, J=5.5 Hz), 3.0-3.1 (2H, m), 3.3-3.4 (1H, m), 3.98 (2H, dd, J=9.0, 5.2 Hz), 4.18 (2H, t, J=8.0 Hz), 6.17 (1H, br s), 7.13 (1H, t, J=7.5 Hz), 7.24 (1H, t, J=7.2 Hz), 7.27 (1H, d, J=7.6 Hz), 7.34 (2H, t, J=7.6 Hz), 7.43 (2H, d, J=7.3 Hz), 7.57 (1H, t, J=7.7 Hz), 7.90 (1H, dd, J=7.9, 1.5 Hz), 11.45 (1H, br s) |
| (2) | 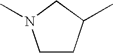 | ¹H NMR (400 MHz, DMSO-d₆: δ): 1.86 (1H, quint., J=10.5 Hz), 2.24 (1H, quint., J=5.7 Hz), 2.6-2.8 (2H, m), 3.01 (1H, quint., J=7.5 Hz), 3.19 (2H, q, J=9.6 Hz), 3.3-3.4 (3H, m), 3.46 (1H, dt, J=10.4, 6.8 Hz), 3.75 (1H, t, J=8.8 Hz), 3.90 (1H, dd, J=10.4, 7.0 Hz), 6.17 (1H, br s), 7.09 (1H, t, J=7.3 Hz), 7.2-7.3 (2H, m), 7.34 (2H, t, J=7.6 Hz), 7.44 (2H, d, J=7.3 Hz), 7.55 (1H, |
| #t, J=7.6 Hz), 7.89 (1H, dd, J=7.9, 1.5 Hz), 11.00 (1H, br s) | | |
| (3) | 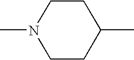 | ¹H NMR (400 MHz, DMSO-d₆: δ): 1.48 (2H, q, J=11.7 Hz), 1.88 (2H, d, J=11.9 Hz), 2.45 (2H, br s), 2.58 (1H, t, J=5.5 Hz), 2.73 (2H, t, J=5.5 Hz), 2.94 (2H, t, J=11.9 Hz), 3.23 (2H, d, J=2.7 Hz), 4.43 (2H, br d, J=13.1 Hz), 6.15 (1H, br s), 7.13 (1H, t, J=7.0 Hz), 7.23 (1H, t, J=7.2 Hz), 7.2-7.4 (2H, m), 7.41 (2H, d, J=7.3 Hz), 7.57 (1H, t, J=7.7 Hz), 7.89 (1H, dd, J=7.9, 2.9 Hz), 11.26 |
| # (1H, br s) | | |
| (4) | 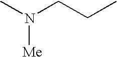 | ¹H NMR (400 MHz, DMSO-d₆: δ): 2.55 (2H, br s), 2.69 (2H, t, J=5.8 Hz), 2.79 (2H, t, J=5.6 Hz), 3.12 (3H, s), 3.23 (2H, d, J=3.0 Hz), 3.69 (2H, t, J=5.6 Hz), 6.14 (1H, br s), 7.08 (1H, t, J=7.5 Hz), 7.2-7.3 (2H, m), 7.33 (2H, t, J=7.8 Hz), 7.42 (2H, d, J=7.2 Hz), 7.5-7.6 (1H, m), 7.85 (1H, dd, J=7.9, 1.5 Hz) |

### EXAMPLE 7

The following compound was prepared in a similar manner to that of Example 4.

(1) 2-[[2-(Dimethylamino)ethyl](methyl)amino]-4(3H)-quinazolinone

¹HNMR(400 MHz, DMSO-d₆: δ): 2.87 (6H, s), 3.22 (3H, s), 3.3-3.4 (2H, m), 3.94 (2H, t, J=5.9 Hz), 7.15 (1H, t, J=7.6 Hz), 7.30 (1H, br), 7.60 (1H, t, J=7.6 Hz), 7.91 (1H, d, J=7.8 Hz)

### EXAMPLE 8

Triethylamine (1.40 mL, 10.0 mmol) was added to a suspension of 2-chloro-4(3H)-quinazolinone (181 mg, 1.00 mmol) and N,N-dimethyl-1,2-ethanediamine (0.196 mL, 1.50 mmol) in dioxane (5 mL), and the mixture was heated at reflux for 2 hours. Cooled to room temperature, and the reaction were quenched with water, and the product was extracted with ethyl acetate. The organic layer was washed with water and dried over sodium sulfate. Purification over silica gel chromatography and treatment of the product with a solution of hydrogen chloride in ethyl acetate (4M, 1 mL) gave 2-{[2-(dimethylamino)ethyl]amino}-4(3H)-quinazolinone hydrochloride (141 mg, 52.3%) as product.

¹H NMR (400 MHz, DMSO-d₆: δ): 2.86 (6H, s), 3.36 (2H, br), 4.00 (2H, br d, J=4.5 Hz), 7.36 (1H, t, J=8.0 Hz), 7.7-7.9 (2H, m), 8.00 (1H, d, J=7.9 Hz), 8.5 (1H, br), 10.46 (1H, br)

1. A compound of the formula (I): 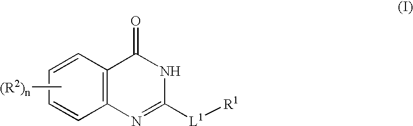
wherein R¹ is substituted cyclic amino groups, optionally substituted carbocyclic group or optionally substituted amino group,
R² is substituent,
n means an integer of 0 to 4, and
L¹ is (1) cyclo(lower)alkylene, (2) cyclo(lower)alkenylene, (3) diradical of saturated- or unsaturated monocyclic group with one or more nitrogen atom (s), which is obtained after removal of one hydrogen atom from said monocyclic group, or (4) —N(R³)—L²— (wherein R³ is hydrogen or lower alkyl, and L² is lower alkylene or lower alkenylene),
or its prodrug, or a salt thereof.

2. The compound according to claim 1, wherein R² is halogen, lower alkyl or lower alkoxy.

3. The compound according to claim 2, wherein R¹ is (1) cyclic amino group substituted with aryl optionally substituted with halogen, lower alkoxy, lower alkyl or halo(lower)alkyl, or (2) lower alkylamino.

4. The compound according to claim 3, wherein R¹ is tetrahydropyridyl, piperidyl or piperazinyl, each of which is substituted with aryl optionally substituted with halogen.

5. The compound according to claim 2, wherein
L is cyclo(lower)alkylene or cyclo(lower)alkenylene.

6. A process for preparing a compound of the formula (I): 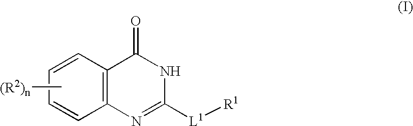
wherein R¹ is substituted cyclic amino groups, optionally substituted carbocyclic group or optionally substituted amino group,
R² is substituent,
n means an integer of 0 to 4, and
L¹ is (1) cyclo (lower) alkylene, (2)
cyclo(lower)alkenylene, (3) diradical of saturated- or unsaturated monocyclic group with one or more nitrogen atom (s), which is obtained after removal of one hydrogen atom from said monocyclic group, or (4) —N(R³)—L²— (wherein R³ is hydrogen or lower alkyl, and L² is lower alkylene or lower alkenylene), or its prodrug, or a salt thereof, which comprises,
(1) subjecting the compound of the following formula (II): 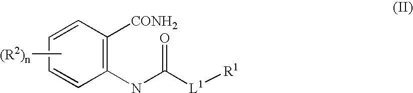
or a salt thereof, to cyclization reaction in the presence of base to provide a compound of the formula (I): 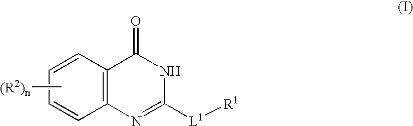
or a salt thereof, in the above formulae, R¹, R², n and L¹ are each as defined above.

7. A pharmaceutical composition which comprises, as an active ingredient, a compound of claim 1 or its prodrug, or a pharmaceutically acceptable salt thereof in admixture with pharmaceutically acceptable carriers or excipients.

8. A compound of claim 1 or its prodrug, or a pharmaceutically acceptable salt thereof for use as a medicament.

9. A pharmaceutical composition of claim 7 for inhibiting PARP activity.

10. A pharmaceutical composition of claim 7 for treating or preventing diseases ascribed by NMDA- and NO-induced toxicity.

11. A pharmaceutical composition of claim 7 for extending the lifespan or proliferative capacity of cells or altering gene expression of senescent cells.

12. A method for treating or preventing tissue damage resulting from cell damage or death due to necrosis or apoptosis; neural tissue damage resulting from ischemia and reperfusion injury, neurological disorders and neurodegenerative diseases; neurodegenerative diseases; head trauma; stroke; Alzheimer's disease; Parkinson's disease; epilepsy; Amyotrophic Lateral Scleosis (ALS); Huntington's disease; schizophrenia; chronic pain; ischemia and nloss following hypoxia; hypoglycemia; ischemia; trauma; nervous insult; previously ischemic heart or skeleton muscle tissue; radiosensitizing hypoxic tumor cells; tumor cells from recovering from potentially lethal damage of DNA after radiation therapy; skin aging; arteriosclerosis; osteoarthritis; osteoporosis; muscular dystrophy; degenerative diseases of skeletal muscle involving replicative senescence; age-related macular degeneration; immune senescence; AIDS; and other immune senescence diseases; inflammatory bowel disorders (e.g., colitis); arthritis; diabetes; endotoxic shock; septic shock; and tumor, which comprises administering a compound of claim 1 or its prodrug, or a pharmaceutically acceptable salt thereof to a human being or an animal.

13. A use of a compound of claim 1 or its prodrug, or a pharmaceutically acceptable salt thereof for the manufacture of a medicament.
